(19) United States
(12) Patent Application Publication
(10) Pub. No.: US 2016/0310038 A1
(43) Pub. Date: Oct. 27, 2016

(54) SYSTEMS AND METHODS OF IMAGE ACQUISITION FOR FOUR DIMENSIONAL MAGNETIC RESONANCE IMAGING

(71) Applicant: Washington University, St. Louis, MO (US)

(72) Inventors: Yanle Hu, St. Louis, MO (US); Dongsu Du, St. Louis, MO (US); Sasa Mutic, St. Louis, MO (US); H. Harold Li, St. Louis, MO (US)

(21) Appl. No.: 15/136,041

(22) Filed: Apr. 22, 2016

Related U.S. Application Data
(60) Provisional application No. 62/152,588, filed on Apr. 24, 2015.

(51) Int. Cl. A61B 5/055; A61B 5/08; A61N 5/10; A61B 5/113

(52) U.S. Cl. CPC A61B 5/055; A61B 5/1135; A61B 5/0816; A61N 5/1039

(57) ABSTRACT

An example magnetic resonance imaging (MRI) system includes a sensor that detects a characteristic amplitude associated with a patient characteristic of a subject, a receiver that receive magnetic resonance data, a memory device, and a processor. The processor is programmed to determine when the characteristic amplitude of the subject reaches a first characteristic amplitude of a plurality of sequential characteristic amplitudes defined for a cycle of the patient characteristic, determine if a predetermined number of MRI images have been acquired for the first characteristic amplitude, capture magnetic resonance data with the receiver if the number of MRI images have not been acquired for the first characteristic amplitude and without regard for a location of the first characteristic amplitude in the sequence of the sequential characteristic amplitudes, and process the magnetic resonance data as an MRI image associated with the first characteristic amplitude.

### CROSS REFERENCE TO RELATED APPLICATIONS

This application claims the priority to U.S. Provisional Patent Application Ser. No. 62/152,588, filed Apr. 24, 2015, the entire disclosure of which is hereby incorporated by reference in its entirety, and for all purposes herein.

### STATEMENT REGARDING FEDERALLY SPONSORED RESEARCH & DEVELOPMENT

This invention was made with government support under grant 1R21CA167092, awarded by the U.S. National Institutes of Health. The U.S. government may have certain rights in the invention.

### BACKGROUND

The embodiments described herein relate generally to magnetic resonance imaging (MRI), and more specifically four dimensional MRI (4DMRI) systems and methods of 4DMRI image acquisition.

When treating abdominal tumors with radiotherapy, respiratory motion is generally taken into consideration. The peak to valley respiratory motion amplitude of a tumor may be more than 10 mm, which can lead to serious degradation in the dose-volume histogram (DVH) of a target tumor. Different treatment strategies have been developed to solve this problem, including larger safety margins, breath holding and treatment machine gating.

Four dimensional (4D) imaging can provide tumor and organ motion information for, among other things, evaluation and selection of treatment strategies. 4D Computed Tomography (4DCT) is sometimes used for such 4D imaging. 4DCT, however, has a relatively low soft tissue contrast and may deliver extra dosage of ionizing radiation to patients. 4DMRI typically has a better tissue contrast than some known systems, and less tumor delineation uncertainty may be achieved. Some known 4DMRI systems capture images, also referred to as slices, based on a patient's respiratory amplitude. The positions of the respiratory cycle at which the MRI machine captures a slice are referred to as triggers.

In an amplitude based trigger system, triggers are often missed during a scan of a patient. Trigger missing happens for at least two reasons. First, the individual slice image acquisition time of the 4DMRI system is often extended to meet a specific absorption rate (SAR) requirement. Therefore, it is probable that a trigger will occur right before the end of previous triggers' image acquisition and will be missed. These missed triggers are referred to as type I missed triggers. Most of the type I missed triggers occur in the middle of an inhalation or an exhalation stage due to the fast changing respiratory amplitude (i.e., fast breathing). The second reason for missed triggers is that the amplitudes of the end of inhalation and the end of exhalation states are typically predefined based on a preparation stage respiratory signal. Because the patient may not always breathe in or out as deeply as during the preparation stage, in some breathing cycles, the breathing depth's amplitude does not reach the predefined end of inhalation or end of exhalation state amplitudes. Because the amplitude never reaches the predefined end of inhalation or end of exhalation, triggers are not detected and images are not captured for the end of inhalation or exhalation during such breathing cycles. These missed triggers are referred to as type II missed triggers. The type II missed triggers predominately occur at the end of inhalation or the end of exhalation states. The respiratory sampling range in an amplitude-based trigger system is the percentage of the full respiratory range between the predefined end of inhalation amplitude and the predefined end of exhalation amplitude. The larger the respiratory sampling range is, the more motion information can be acquired from the captured 4D images. However, increasing the respiratory sampling range will result in more type II missed triggers.

In some known systems and methods, the slice acquisition order is prefixed. If a trigger is missed, the scanner waits until the respiratory signal reaches the missed trigger position again. That is, if a trigger is missed during the exhalation state, the scanner waits until that trigger for the exhalation state is captured before collecting additional slices. The waiting time is referred to as a scan halt. Scan halts extend the scan time, and in some cases significantly extend the scan time. Scan halts can be reduced by reducing the missed triggers. An improved order prefixed acquisition method splits the respiratory states into multiple groups to decrease the amount of triggers in each respiratory cycle. The type I missed triggers may be reduced by using this method, while the type II missed triggers still occur. The scan time of using either of these methods may still be too long for some clinical use, especially when the respiratory sampling range is large.

### BRIEF DESCRIPTION

One aspect of the disclosure is an example magnetic resonance imaging (MRI) system including a sensor that detects a characteristic amplitude associated with a patient characteristic of a subject, a receiver that receive magnetic resonance data, a memory device, and a processor. The processor is programmed to determine when the characteristic amplitude of the subject reaches a first characteristic amplitude of a plurality of sequential characteristic amplitudes defined for a cycle of the patient characteristic, determine if a predetermined number of MRI images have been acquired for the first characteristic amplitude, capture magnetic resonance data with the receiver if the number of MRI images have not been acquired for the first characteristic amplitude and without regard for a location of the first characteristic amplitude in the sequence of the sequential characteristic amplitudes, and process the magnetic resonance data as an MRI image associated with the first characteristic amplitude.

In another aspect, an example method for acquiring a predetermined number of magnetic resonance imaging (MRI) images at each of a plurality of sequential characteristic amplitudes of a subject for four dimensional (4D) MRI is provided. Each characteristic amplitude is an amplitude within a cycle of a patient characteristic of a subject. The method is at least partially performed by a processor. The method includes monitoring the characteristic amplitude of the subject, detecting when the monitored characteristic amplitude reaches a first characteristic amplitude of the plurality of sequential characteristic amplitudes, capturing magnetic resonance data for the first characteristic amplitude if the predetermined number of MRI images have not been acquired for the first characteristic amplitude and without regard for a location of the first characteristic amplitude in the sequence of the sequential characteristic amplitudes, and processing the magnetic resonance data as an MRI image associated with the first characteristic amplitude.

In yet another aspect, at least one non-transitory computer-readable storage media having computer-executable instructions embodied thereon is provided. When executed by at least one processor, the computer-executable instructions cause the processor to determine when a characteristic amplitude of a subject reaches a first characteristic amplitude of a plurality of sequential characteristic amplitudes defined for a cycle of a patient characteristic of the subject, determine if a predetermined number of MRI images have been acquired for the first characteristic amplitude, capture magnetic resonance data if the predetermined number of MRI images have not been acquired for the first characteristic amplitude and without regard for a location of the first characteristic amplitude in the sequence of the sequential characteristic amplitudes, and process the magnetic resonance data as an MRI image associated with the first characteristic amplitude.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. As the color drawings are being filed electronically via EFS-Web, only one set of the drawings is submitted.

FIG. 1 is a simplified example MRI system that may be used for amplitude triggered 4DMRI acquisition.

FIG. 2 is a flow diagram of an example embodiment of a greedy acquisition method for use in the MRI system shown in FIG. 1.

FIG. 3 is a flow diagram of an example embodiment of a greedy acquisition method for use in the MRI system shown in FIG. 1.

FIG. 4 is an example graph of the trigger positions on an example respiratory signal in a prior art order prefixed acquisition method.

FIG. 5 is an example graph of the trigger positions on an example respiratory signal in a prior art improved order prefixed acquisition method.

FIGS. 6A-6D are graphs of the triggers on a respiratory signal from an example experiment for the order prefixed acquisition method show in FIG. 4.

FIGS. 7A-7D are graphs of the triggers on a respiratory signal from an example experiment for the improved order prefixed acquisition method shown in FIG. 5.

FIGS. 8A-8D are graphs of the triggers on a volunteer's respiratory signal from an example experiment for the greedy acquisition method.

### DETAILED DESCRIPTION

The embodiments described herein relate generally to magnetic resonance imaging (MRI). More specifically, the embodiments described herein relate generally to four dimensional MRI (4DMRI) systems and methods of 4DMRI image acquisition using a greedy acquisition method.

As used herein, a processor such as the processor 120 may include any programmable system including systems using micro-controllers, reduced instruction set circuits (RISC), application specific integrated circuits (ASICs), logic circuits, and any other circuit or processor capable of executing the functions described herein. The above examples are example only, and are thus not intended to limit in any way the definition and/or meaning of the term “processor.”

As described herein, computing devices and computer systems include a processor and a memory. However, any processor in a computer device referred to herein may also refer to one or more processors wherein the processor may be in one computing device or a plurality of computing devices acting in parallel. Additionally, any memory in a computer device referred to herein may also refer to one or more memories wherein the memories may be in one computing device or a plurality of computing devices acting in parallel.

An example 4DMRI system includes an MRI device, a receiver, and a controller for operating and monitoring the MRI device. The MRI device outputs a radio frequency (RF) signal (also referred to as “magnetic resonance data”) to the receiver that, when processed by the controller, generates image data to be displayed to a user. The controller is configured to monitor, or scan, for a patient characteristic such as respiratory amplitude. The user or the controller defines for the receiver a set of values of the characteristic to monitor. When the controller detects the characteristic having a value within the set of values, the receiver triggers, or captures, the output signal for the controller to process as image data. A slice refers to a two dimensional image from the output signal of a portion of a three dimensional (3D) object (e.g., a patient) captured at each trigger. In other embodiments, a slice refers to a plurality of MRI images grouped together as a 3D portion of a 3D object.

A greedy acquisition method is used by the 4DMRI system. The greedy acquisition method is an acquisition method that is defined by a first come, first triggered principle. Before scanning is initiated, a number of slices and trigger positions (i.e., the set of values for the characteristic) are defined by the user and/or the controller. In the event of a missed trigger, the 4DMRI system does not wait for an identical trigger position in a subsequent breathing cycle before continuing to scan for other triggers. Rather, the greedy acquisition method triggers at the next available trigger that has not had all of its desired slices captured. The 4DMRI system monitors the number of slices collected at each trigger position until all slices at each trigger position have been collected.

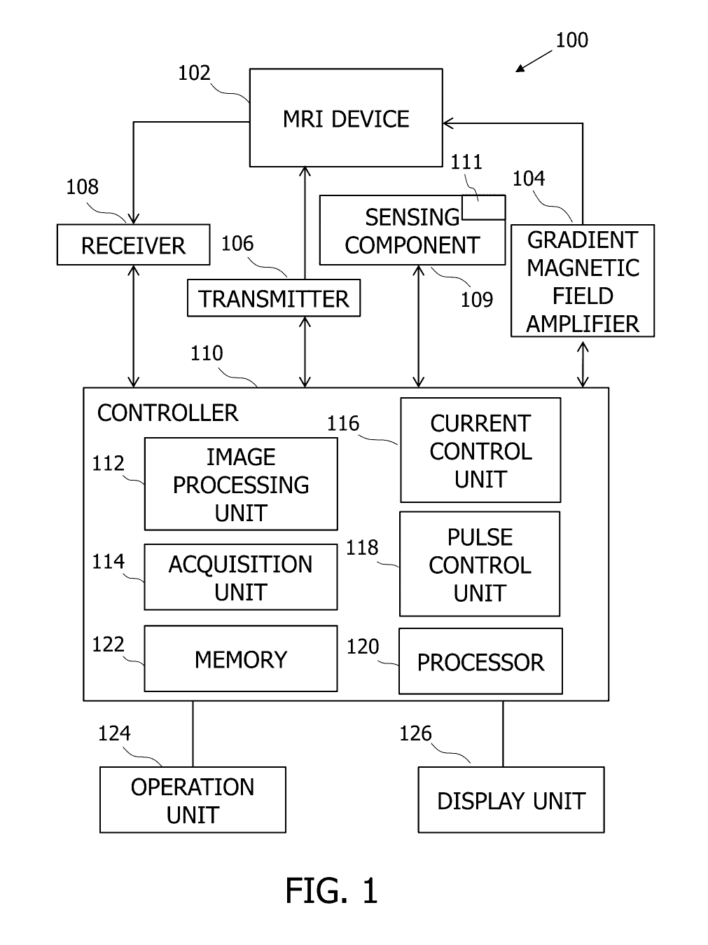

FIG. 1 is a simplified example MRI system 100. In example embodiment, the MRI system 100 is a 4DMRI system. The MRI system 100 includes a MRI device 102, a gradient magnetic field amplifier 104, a transmitter 106, a receiver 108, and a controller 110. In some embodiments, the components of the MRI system 100 may be combined and/or separated in an alternative design to FIG. 1. The MRI system 100 may include additional components configured to provide support and/or additional capabilities for the components shown in FIG. 1.

The MRI device 102 includes at least one magnet (not shown) coupled to a plurality of coils (not shown). In the example embodiment, the MRI device includes one magnet with a bore to house a patient (not shown). In some embodiments, where the MRI device 102 includes at least two magnets, the patient is positioned between at least one pair of magnets. The coils include a superconductive coil to produce a static magnetic field, a RF coil to produce a RF pulse, three gradient coils to produce a gradient magnetic field along each axis of the x-y-z grid, and a receiving coil to capture the output signals of the MRI device 102.

The gradient magnetic field amplifier 104 is coupled to each gradient coil of the MRI device 102. The gradient magnetic field amplifier 104 outputs an amplified gradient magnetic field signal to the gradient coils to induce the gradient magnetic fields. The transmitter 106 is coupled to the RF coil to supply current to the RF coil of the MRI device 102 to generate RF pulses.

The receiver 108 is coupled to the receiving coil to process the output signal or magnetic resonance data of the MRI device 102. The controller 110 scans (e.g., via a motion sensor, location sensor, scanning of the output signal image, etc.) for a patient characteristic, such as respiratory amplitude. When a characteristic amplitude or magnitude of the patient characteristic is detected at a predefined value (i.e., a trigger), the receiver 108 captures the output signal to be sent to the controller 110 for image processing. In some embodiments, the receiver 108 sends the characteristic data with the output signal to the controller 110. In some embodiments, a sensing component 109 (e.g., a respiratory belt) that includes a sensor 111 is configured to monitor and detect characteristic amplitudes of the patient characteristic and cause the receiver 108 to capture the output signal.

In an example, the component 109 is a respiratory belt is coupled to the subject such that the respiratory belt 109 engages the body of the subject. The respiratory belt 109 may be inflated or otherwise provide a pressure against the subject. This pressure is variable as a function of the subject's respiratory cycle. A pressure sensor 111 of the respiratory belt 109 collects sensor data associated with the pressure to determine a respiratory amplitude of the subject. The sensor data is transmitted to the receiver 108 and/or the controller 110 to determine when the respiratory amplitude has reached a trigger value.

The controller 110 is in communication with the gradient magnetic field amplifier 104, the transmitter 106, and the receiver 108 to send and receive data (e.g., image data) and control information (e.g., current monitoring). In some embodiments, the controller 110 is in communication with the MRI device 102 to monitor and/or control the operation of the MRI device 102.

The controller 110 includes an image processing unit 112, an acquisition unit 114, a current control unit 116, a pulse control unit 118, a processor 120, and a memory 122. In the example embodiment, the controller 110 is a computing device further including an operation unit 124 and a display unit 126. The at least one processor of the controller 110 may include the image processing unit 112, the acquisition unit 114, the current control unit 116, and/or the pulse control unit 118.

The image processing unit 112 is configured to receive the magnetic resonance data from the receiver 108 and processes the data as an MRI image to be displayed. In the example embodiment, the image processing unit 112 measures patient characteristic data such as respiratory amplitude to be used by the acquisition unit 114. Additionally, the image processing unit 112 may provide additional display options at the display unit 125 such as three dimensional (3D) models of images and time-lapsed videos.

The acquisition unit 114 is configured to control the triggering function of the receiver 108 and determine if an image produced by the image processing unit 112 is to be stored in the memory 122. In the example embodiment, the acquisition unit 114 is configured to operate using a greedy acquisition method as described in detail below.

The current control unit 116 regulates the input current of the gradient magnetic field amplifier 104 and the transmitter 106 to facilitate currents within the current ratings of each component. In some embodiments, the current control unit monitors electric current data received from the MRI system 100.

The pulse control unit 118 is in communication with the transmitter 106 to generate RF pulses within the MRI device 102 to create the output signal received by the receiver 108. In some embodiments, the pulse control unit 118 is in communication with the gradient magnetic field amplifier 104 to monitor and/or control the gradient magnetic field signals sent to the MRI device 102.

Processor 120 may include any type of conventional processor, microprocessor, or processing logic that interprets and executes instructions. Processor 120 can process instructions for execution within the controller 110, including instructions stored in the memory 122 to display graphical information for a GUI on an external input/output device, such as display 126 coupled to a high speed interface. In other implementations, multiple processors and/or multiple buses may be used, as appropriate, along with multiple memories and types of memory. Also, multiple controllers 110 may be connected, with each device providing portions of the necessary operations (e.g., as a server bank, a group of blade servers, or a multi-processor system). In some embodiments, the processor 120 may include the image processing unit 112, the acquisition unit 114, the current control unit 116, and/or the pulse control unit 118.

The memory 122 facilitates data storage in the MRI system 100. In some embodiments, the memory 122 includes a plurality of storage components such as, but not limited to, a hard disk drive, flash memory, random access memory, and a magnetic or optical disk. Alternatively or additionally, the memory 122 may include remote storage such a server in communication with the controller 110. The memory 122 stores at least one computer program that, when received by the at least one processor, cause the at least one processor to perform any of the functions of the controller 110 described above. In one implementation, the memory 122 may be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product may also contain instructions that, when executed, perform one or more functions, such as those described herein. The information carrier is a non-transitory computer- or machine-readable medium, such as the memory 122 or memory on the processor 120. Additionally, the memory 122 is configured to facilitate storage of a plurality of images from the MRI device 102 as processed by the controller 110.

The operation unit 124 enables a user to interface (e.g., visual, audio, touch, button presses, stylus taps, etc.) with the controller 110 to control the operation of the MRI system 100. In some embodiments, the operation unit 124 is further coupled to the MRI device 102 to control the operation of the MRI device 102.

The display unit 126 enables a user to view data and control information of the MRI system 100. The display unit 126 may further be coupled to other components of the MRI system 100 such as the MRI device 102. The display unit 126 may include a visual display such as a cathode ray tube (CRT) display, liquid crystal display (LCD), light emitting diode (LED) display, or “electronic ink” display. In some embodiments, the display unit 315 is configured to present a graphical user interface (e.g., a web browser and/or a client application) to the user. A graphical user interface may include, for example, an image display for images acquired by the MRI system 100 of a patient, operational data of the MRI system 100, and the patient's physiological data (e.g., heart rate).

The MRI system 100 may be used to capture 4DMRI images using a greedy acquisition method. The MRI system 100 uses amplitude triggers to determine when to capture a slice. In relation to MRI scanning techniques that may be used in the MRI system 100, respiratory states are discrete, sequential points of the respiratory cycle of a patient that trigger the receiver 108 to capture a slice or image of the patient. The respiratory states are determined according to the acquisition method used by the acquisition unit 114 and/or the user's requirements. In 4DMRI acquisition, a plurality of images is acquired at each respiratory state. In the example embodiment, each image may be referred to as a slice. In other embodiments, a slice may refer to a plurality of images grouped together. By capturing a plurality of slices at each respiratory state, each slice can be checked for accuracy against similar slices. For example, the slices may be compared to each other to define an accuracy threshold. Slices within the threshold are considered to be accurate for the respiratory state. Slices that are outside of the threshold are considered to be inaccurate and are removed from analysis. For amplitude triggered 4DMRI, the respiratory states are predetermined based on respiratory amplitudes and stages (i.e., inhalation and exhalation). The respiratory states are uniformly sampled between the end of inhalation and the end of exhalation states. In the example experiment described below, the respiratory states are 0%, 20% ↑, 40% ↑, 60% ↑, 80% ↑, 100%, 80% ↓, 60% ↓, 40% ↓, and 20%↓. 0% represents the end of exhalation state and 100% represents the end of inhalation state. The ‘↑’ and ‘↓’ symbols represent the inhalation stage and the exhalation stage, respectively. The respiratory states are identified in a predetermined sequential order of the respiratory cycle.

In order to reduce the 4DMRI acquisition duration, in particular with respect to delays from scan halts caused by missed triggers, the optimization problem in Equation 1 with the constraints in Equations 2-10 is presented. In the equations, t_{sd} is the time of slice s of S number of slices at respiratory state d of D number of respiratory states is presented. By reducing t_{sd}, the total scan time for a patient may be reduced. Equation 1 is:

$$\begin{matrix}Minimize max ( {t}_{sd} ) d = 1 , … , D ; s = 1 , … , S \\ Subject to \text{:} & ( 1 ) \\ {A}_{step} = \frac{{A}_{eoi} - {A}_{eoe}}{D / 2} \\ and \text{:} & ( 2 ) \\ {A}_{tot} = ( {A}_{eoi} - {A}_{eoe} ) * tol & ( 3 )\end{matrix}$$

Aₑₒᵢ and Aₑₒₑ of Equations 2 and 3 represent the end of inhalation and the end of exhalation state trigger amplitudes, respectively. Aₑₒᵢ and Aₑₒₑ are pre-calculated in the preparation stage. Aₛₜₑₚ is the trigger amplitude difference between adjacent respiratory states. Aₜₒₗ is the trigger tolerance of a percentage tol of the respiratory sampling range (Aₑₒᵢ−Aₑₒₑ). In some embodiments, tol is equal to 1% to 2%.

Equations 4-7 are used to facilitate triggering slice s at the correct amplitude and stage of state d. The respiratory signal is represented by ƒ(x). With respect to Equations 6 and 7 below, Equations 4 and 5 define the inhalation and exhalation states for dε[1, D]. The respiratory states d=1 and d=D/2+1 represent transitional respiratory states between the inhalation and exhalation stages. The inhalation stage is defined by:

$$stage ( {t}_{sd} ) = inhalation for d ∈ [ 2 , \frac{D}{2} ] \qquad ( 4 )$$

The exhalation stage is defined by:

$$stage ( {t}_{sd} ) = exhalation for d ∈ [ \frac{D}{2} + 2 , D ] \qquad ( 5 )$$

For dε[1, D/2] (i.e., the end of exhalation stage and the inhalation stage), the respiratory signal is represented by:

ƒ(t_{sd})ε[Aₑₒₑ+Aₛₜₑₚ*(d−1)−Aₜₒₗ,Aₑₒₑ+Aₛₜₑₚ*(d−1)+Aₜₒₗ] (6)

For dε[(D/2)+1, D] (i.e., the end of inhalation stage and the exhalation stage), the respiratory signal is represented by:

ƒ(t_{sd})ε[Aₑₒₑ+Aₛₜₑₚ*(D+1−d)−Aₜₒₗ,Aₑₒₑ+Aₛₜₑₚ*(D+1−d)+Aₜₒₗ] (7)

Equation 8 eliminates the overlap of image acquisitions, where Tₛₕₒₜ is the acquisition time of each slice. Equation 8 is defined as:

abs(tₛ_{₁}_{d}_{₁}−tₛ_{₂}_{d}_{₂})≧Tₛₕₒₜ (8)

Equation 9 ensures that the time difference between the acquisitions of the slices s at different respiratory states (d₁ and d₂), or effective repetition time (TR), are within a specific range ([T_{low}, T_{high}]) for contrast formation (e.g. [6 s, ∞] for T₂ weighting), where Equation 9 is:

abs(t_{sd}_{₁}−t_{sd}_{₂})ε[T_{low},T_{high}] (9)

Equation 10 is the constraint to reduce slice cross talk, where SSₘᵢₙ is the minimum number of slice separation between any adjacent acquisitions. In the example experiment described below, SSₘᵢₙ is set to 4. So if the current trigger initiates image acquisition from slice 10, for the immediate subsequent valid trigger, slice 7, 8, 9, 10, 11, 12 and 13 should be excluded from image acquisition. Equation 10 is:

abs(s₁−s₂)≧SSₘᵢₙ for any adjacent acquisitions (10)

A greedy acquisition method is used with the MRI system 100 to satisfy Equation 1 with the constraints Equations 2-10. The greedy acquisition method facilitates reducing and/or eliminating scan halts by treating each slice individually. The greedy acquisition method follows a first come first triggered principle to acquire slices. As described further below, in the event of a missed trigger, the greedy acquisition method does not cause the MRI system 100 to wait until a trigger identical to the missed trigger is captured before continuing to acquire further slices (i.e., causing a scan halt). Rather, the greedy acquisition method causes the MRI system 100 to automatically continue acquiring slices for each respiratory state as encountered without regard for a location of the respiratory state in the sequence of respiratory states until a desired, predetermined number of slices for each respiratory state is acquired. Once the predetermined number of slices has been acquired for a particular respiratory state, the MRI system 100 prevents additional slice to be acquired for that respiratory state.

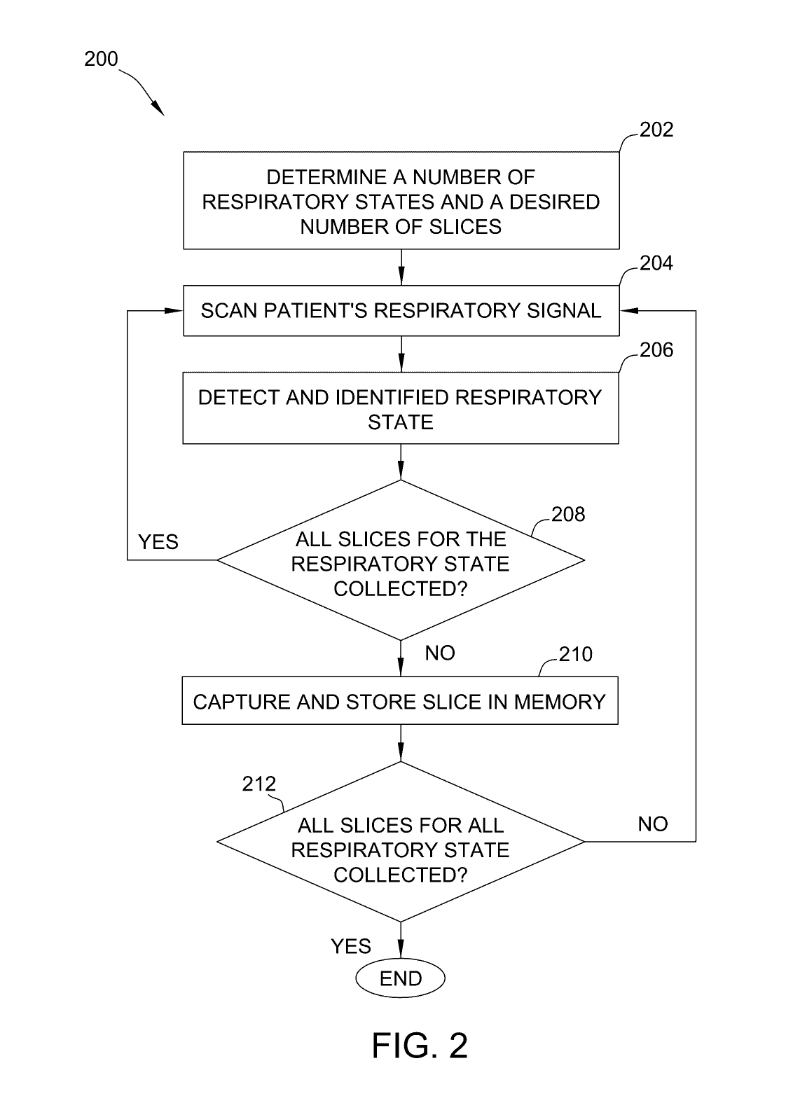

FIG. 2 is a flow diagram 200 of an example greedy acquisition method within the example amplitude-triggered 4DMRI system as shown in FIG. 1. A number of respiratory states and a desired number of slices per respiratory state are determined 202 from a patient's respiratory cycle and a user's (e.g., a doctor) requirements. The MRI system 100 begins to scan 204 the patient and detects 206 at an initial identified respiratory state. In the example embodiment, the initial respiratory state is either the end of exhalation state or the end of inhalation state after a waiting period. The waiting period facilitates avoiding transient signal output from the MRI device 102. In other embodiments, the initial respiratory state is the first respiratory state that the MRI system 100 detects. The controller 110 checks 208 within the memory 122 to determine if the current respiratory state has reached the desired number of slices stored. If the current respiratory state has already reached the identified number of slices, the controller 110 does not signal the receiver 108 to capture the slice and the MRI system 100 continues scanning 204 for the next identified respiratory state.

If the current respiratory state has not reached the desired number of slices acquired, the controller 110 signals the receiver 108 to capture 210 and store the current slice in the memory 122 such that the respiratory state is identifiable. In the example embodiment, a Boolean value representing the acquisition status for each slice is stored within the memory 122. In some embodiments, the captured slice is combined with a respiratory state identifier (e.g., a header with respiratory state information) in the memory 122. In other embodiments, the captured slice is stored within a specific location within the memory 122 for the respiratory state (e.g., an array). Additionally or alternatively, the MRI machine may include a global counter for each respiratory state.

The controller 110 checks 212 the memory 122 to determine if all the slices for all the respiratory states have been acquired. If all the slices have been acquired, the acquisition ends. Otherwise, the MRI system 100 continues scanning 204 to capture the remaining slices.

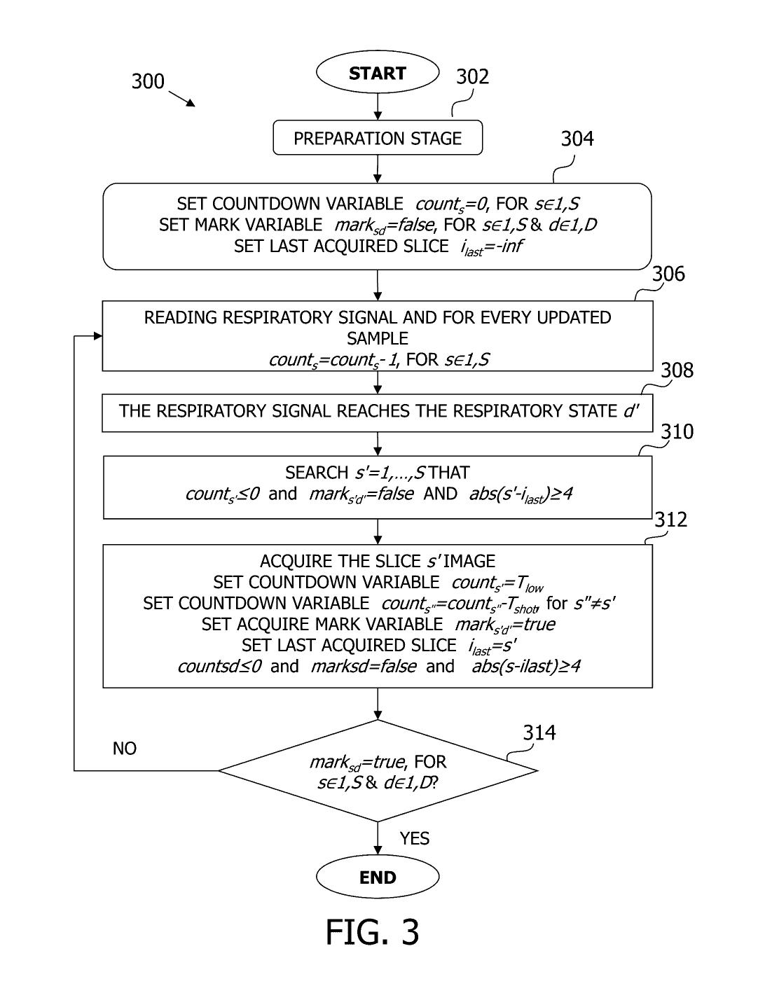

FIG. 3 is a further flow diagram describing another example greedy acquisition method 300 within an amplitude triggered 4DMRI system (e.g., the MRI system 100 shown in FIG. 1). The 4DMRI system initializes 302 at a preparation stage, where the preparation stage includes determining a desired number of slices S to be acquired and a number of respiratory states D. The 4DMRI system initializes 304 for each slice s′ a countdown variable countₛ and a mark variable mark_{sd} (i.e., the slice has been acquired). The countdown variable countₛ facilitates satisfying Equations 8 and 9 as described below. In other embodiments, a counter variable that increments is used to satisfy Equations 8 and 9. A last acquired slice variable iₗₐₛₜ is also initialized.

The 4DMRI system reads 306 the respiratory signal of a patient while decrementing the countdown variable countₛ. The 4DMRI system reaches 308 a respiratory state d′. The 4DMRI system determines 310 that for at least one slice s′, the countdown variable count_{s′} has expired (or is less than or equal to 0), the mark variable mark_{s′d′} indicates a slice has not been acquired, and the slice s′ satisfies Equation 10. In the example embodiment, SSₘᵢₙ is equal to 4. If at least one slice s′ satisfies the above conditions, the slice is acquired 312. In other embodiments, other conditions to determine slice acquisition may be used to in addition to and/or in place of the described conditions. If no slices satisfy the conditions, the 4DMRI system skips the current respiratory state and continues to read 306 the respiratory signal.

While the slice s′ is acquired 312, the countdown variable for the slice s count_{s′} is set equal to T_{low} to facilitate satisfying Equation 9. The countdown variable for other slices count_{s″} is set equal to count_{s″}−Tₛₕₒₜ because during the Tₛₕₒₜ acquisition period, respiratory signal is no longer updated. The current slice s′ is marked as acquired and set as the last acquired slice. The 4DMRI system checks 314 if all of the slices have been acquired. If all slices have been acquired, the greedy acquisition method 300 ends. If at least one slice has not been acquired, the 4DMRI system continues to read 306 the respiratory signal until all slices have been acquired.

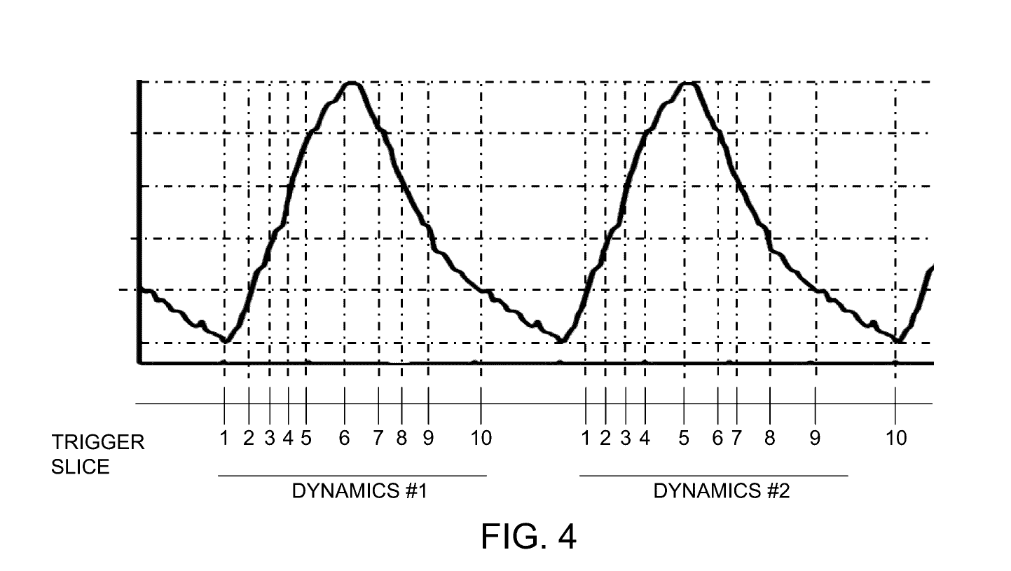

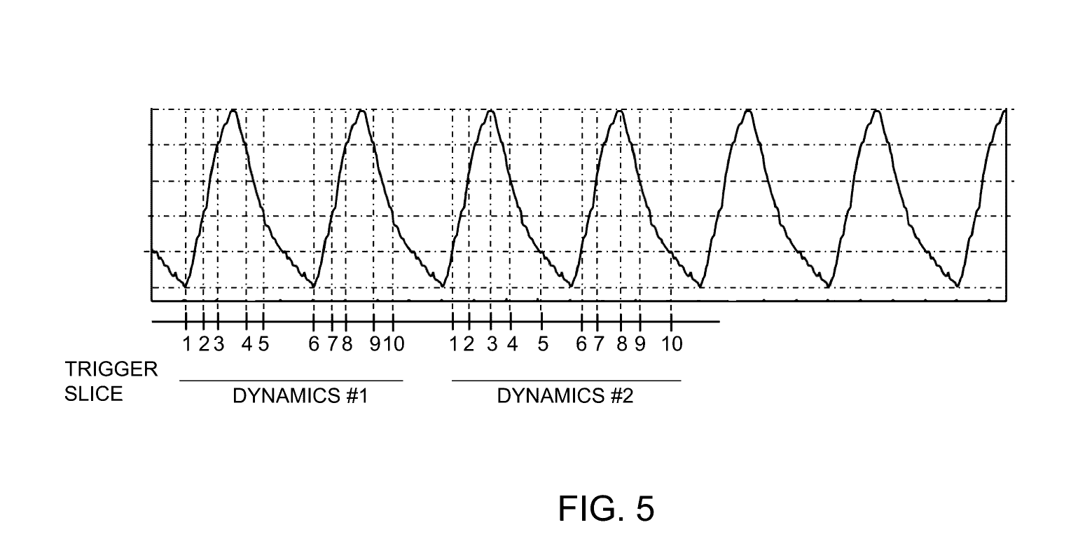

An example experiment comparing the scan time of a 4DMRI system using the greedy acquisition method to the scan times of systems using two other known acquisition methods. The two known acquisition methods are an order prefixed acquisition method and an improved order prefixed acquisition method. FIG. 4 is a graph of an example order prefixed acquisition method used to capture 10 slices per respiratory cycle. FIG. 5 is a graph of an example improved order prefixed acquisition method used to capture 10 slices of a respiratory cycle using two groups of triggers over two respiratory slices. Both methods acquire slices sequentially, such that a missed trigger will cause the MRI machine to wait until an identical trigger is acquired before continuing. For example, if trigger 4 is missed, the system will wait without capturing any images through triggers 5-10 in the current respiratory cycle and triggers 1-3 in the next respiratory cycle in order to capture an image at trigger 4 of the next respiratory cycle. The improved order prefixed acquisition method splits the respiratory states into several groups in an interleaved manner and the different groups are acquired separately such that the number of slices that are acquired in a single respiratory cycle is reduced. In FIG. 5, slices 1-5 are captured in one respiratory cycle and slices 6-10 are captured in a proceeding respiratory cycle.

In the example experiment, three healthy volunteers' respiratory signals were recorded with a commercial respiratory belt that utilized an air filled cushion with a pressure sensor. For each volunteer, approximately 10 minutes of the volunteer's respiratory signal was acquired. For simulation purposes, each volunteer's respiratory signal was extended by repeating it several times. The original respiratory signal contained more than 150 breathing cycles; therefore the repetition did not disturb the signal's statistical property.

Because T2 weighted images show a better tumor contrast, the example experiment mainly focused on T2 weighted 4DMRI in the simulation. Considering an average person's size, the in-plane field of view (FOV) was set at 375 mm*260 mm. The in-plane spatial resolution was set to 1.5 mm*1.5 mm. The slice number was 60. Parallel imaging (acceleration factor=2.0 for TSE) and partial-k acquisition (k-space coverage=0.700) techniques were used to reduce total acquisition time. A single slice scan time was 380 ms (Tₛₕₒₜ=380 ms), including 250 ms image acquisition time and 130 ms pure waiting time. In order to acquire T2 weighted images, TR=6000 ms was used.

In the example experiment, to balance the clinical usability and total scan time, 10 respiratory states were divided to equally sample the respiratory cycle based on the amplitude and the stage (0%, 20% ↑, 40% ↑, 60% ↑, 80% ↑, 100%, 80% ↓, 60% ↓, 40% ↓, and 20% ↓). The trigger tolerance was 1% of the difference between the end of inhalation amplitude and the end of exhalation amplitude. The acquisition process was simulated on the real respiratory signals using MATLAB (MathWorks, Newton, Mass.). The total scan time of different methods on the same volunteer's respiratory signal were compared.

The averaged local maximum signal and the averaged local minimum signal inside a 20 seconds preparation stage were used as the initial end of inhalation stage and end of exhalation stage amplitudes. In addition, the respiratory sampling range was varied to different levels and the total scan time was recoded. The greedy acquisition method was compared with the order prefixed acquisition methods under different respiratory sampling ranges.

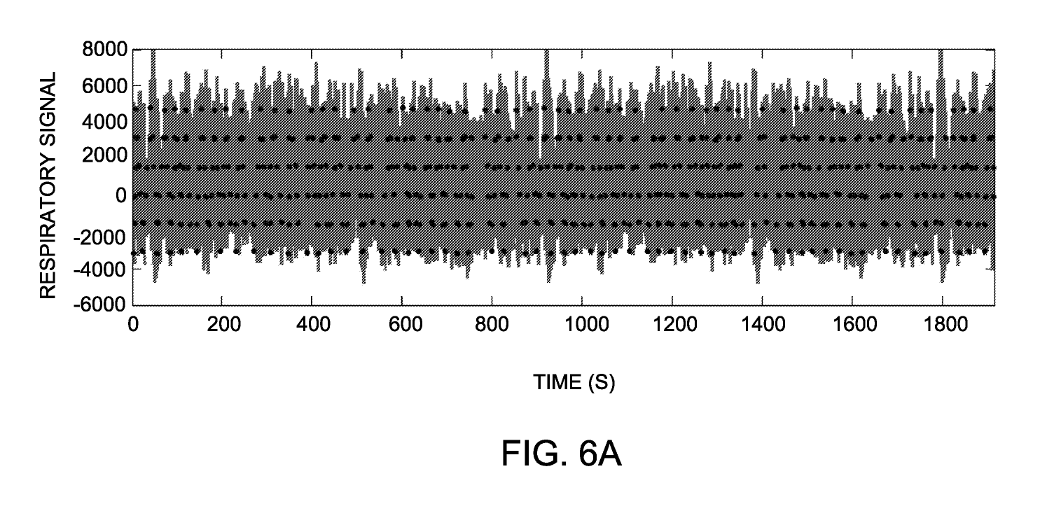

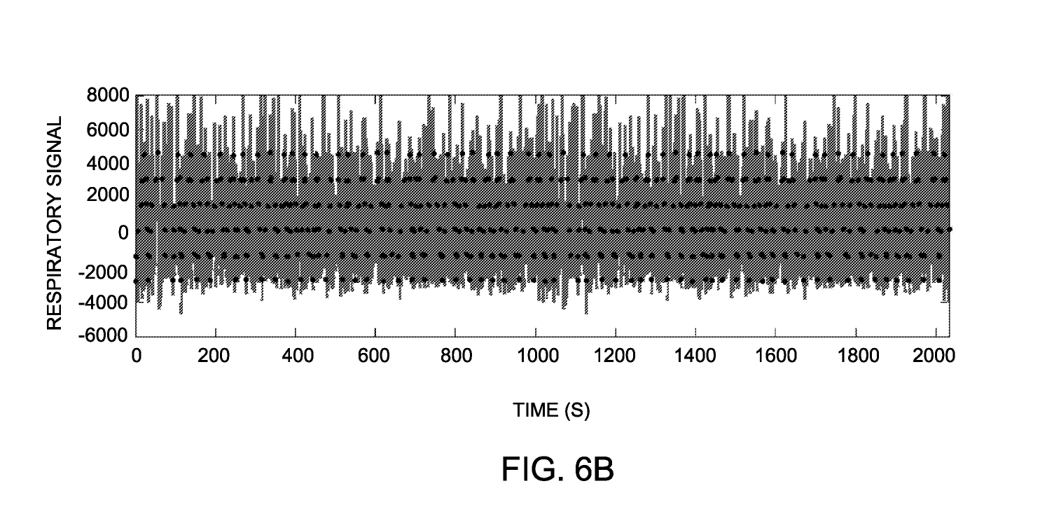

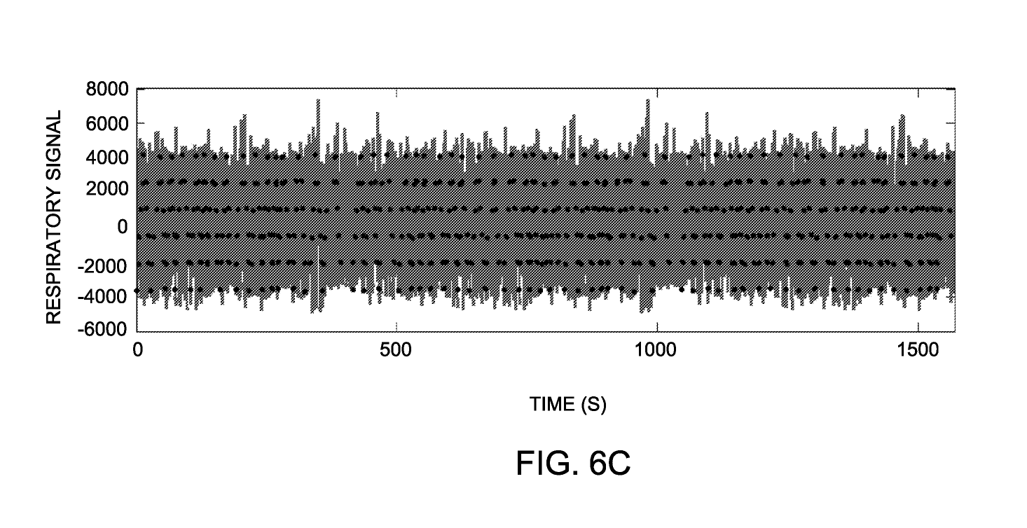

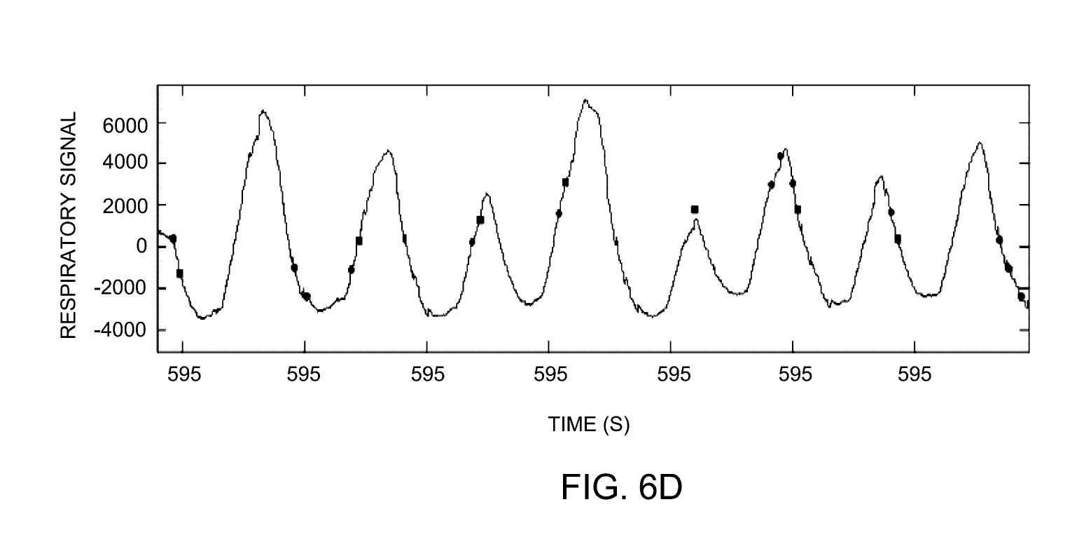

FIGS. 6A-6D show the acquisition simulation results of the order prefixed acquisition method in the example experiment. FIGS. 6A-6C show the triggering process in each of the three healthy volunteers. Each dot represents an image acquisition trigger position (i.e., the trigger positions that have been triggered) and the waveform is the volunteer's respiratory signal. The end of inhalation and the end of exhalation state amplitudes were calculated using 20 seconds preparation stage signals. FIG. 6D shows a subset of FIGS. 6A-6C to further illustrate missed triggers within the order prefixed acquisition method. Each circle is a successful trigger. Each square represents a missed trigger. As described above, the order prefixed acquisition method does not trigger again after a missed trigger until an identical trigger is acquired (e.g., the missed trigger at approximately 611 seconds causes a scan halt until an identical trigger is captured at approximately 619 seconds). The total scan time of the three volunteers was 1914 s, 2029 s and 1562 s respectively for the order prefixed acquisition method (excluding the scanner adjustment and respiration preparation stages).

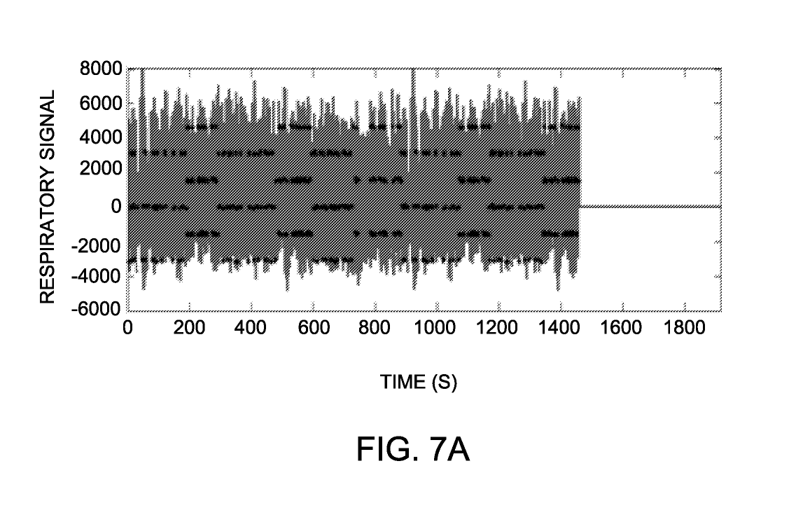

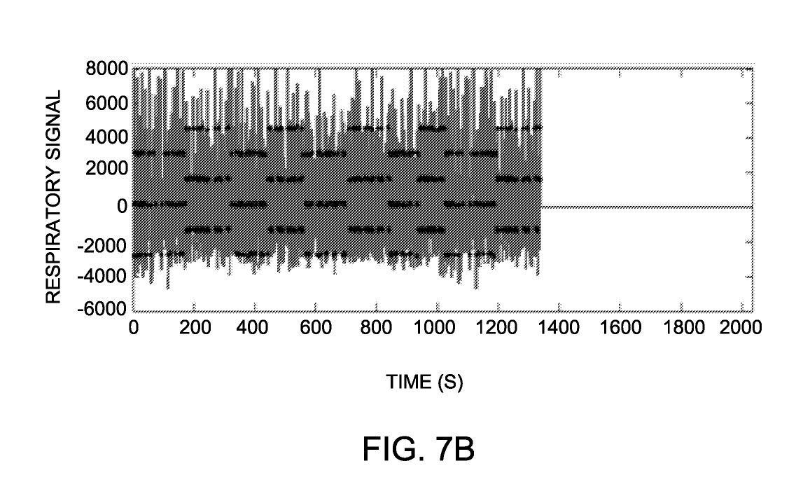

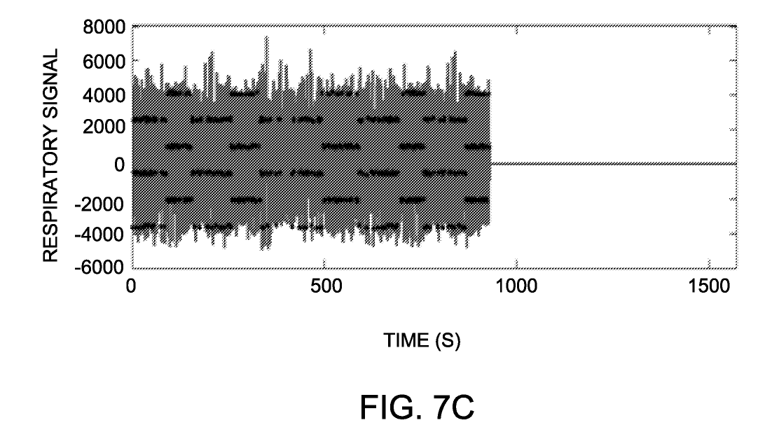

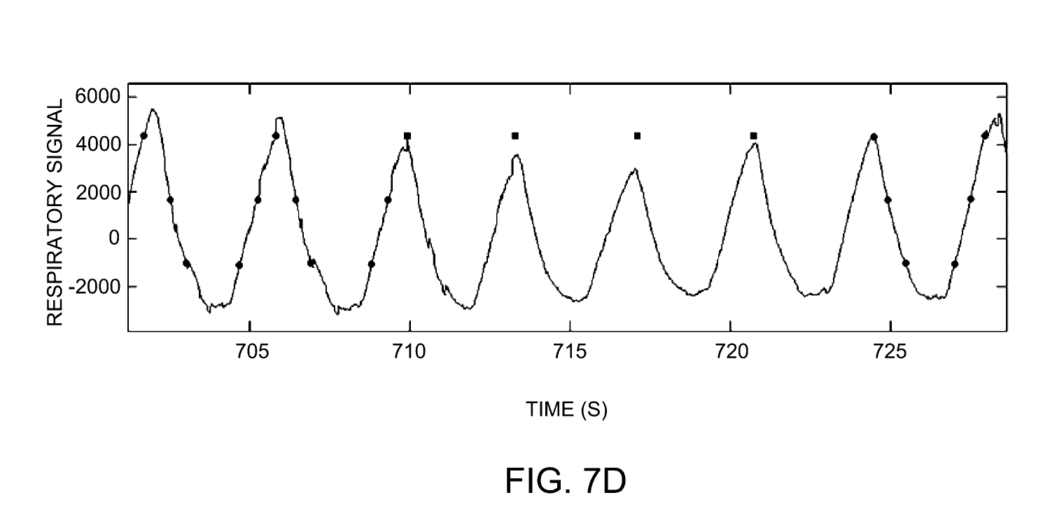

Similar to FIGS. 6A-6D, FIGS. 7A-7D show the acquisition simulation results of the improved order prefixed acquisition method in the example experiment. FIGS. 7A-7C show the triggering process in each of the three healthy volunteers. Each dot represents an image acquisition trigger position and the waveform is the volunteer's respiratory signal. As with the order prefixed acquisition method in FIGS. 6A-6D, the end of inhalation and the end of exhalation state amplitudes were calculated using 20 seconds preparation stage signals. FIG. 7D shows a subset of FIGS. 7A-7C to further illustrate missed triggers within the improved order prefixed acquisition method. Each circle is a successful trigger. Each square represents a missed trigger. The total scan time of the three volunteers was 1459 s, 1337 s, and 929 s respectively for the improved order prefixed acquisition method (excluding the scanner adjustment and respiration preparation stages).

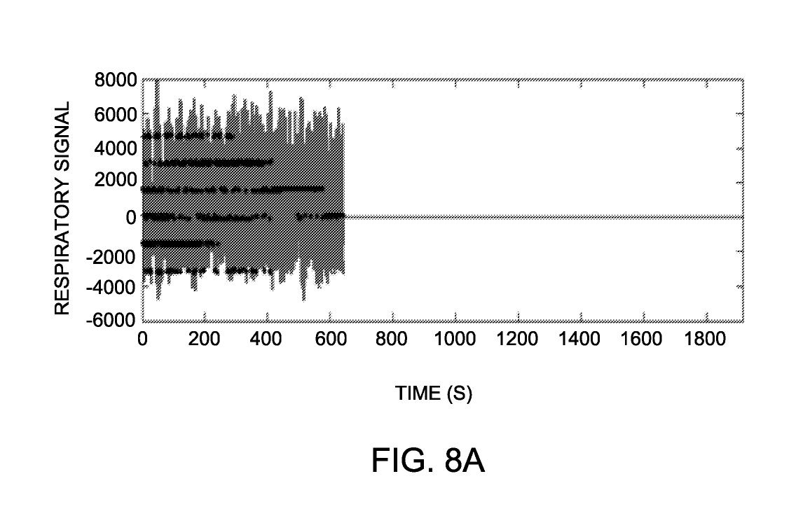

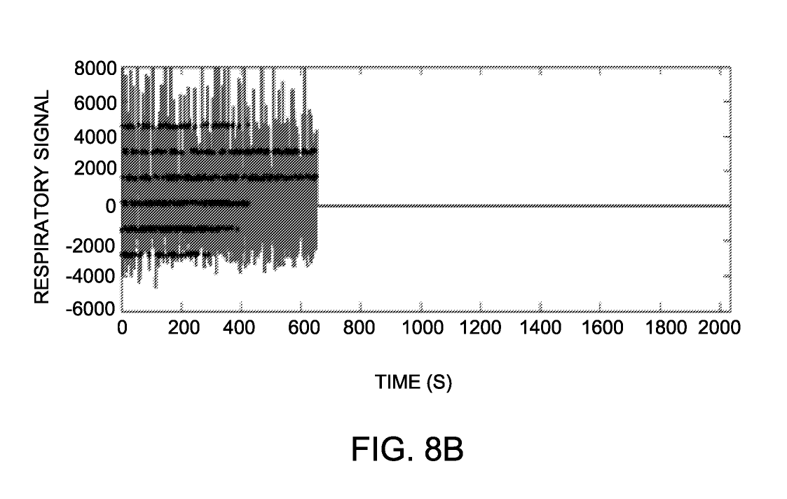

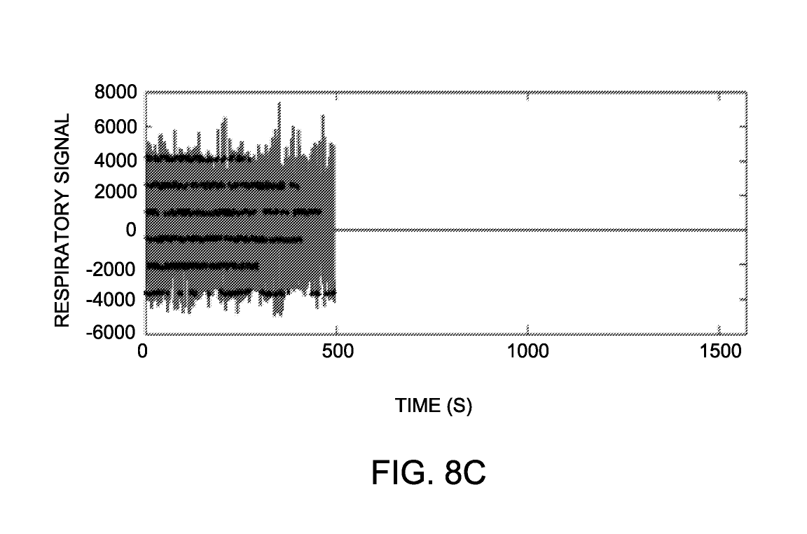

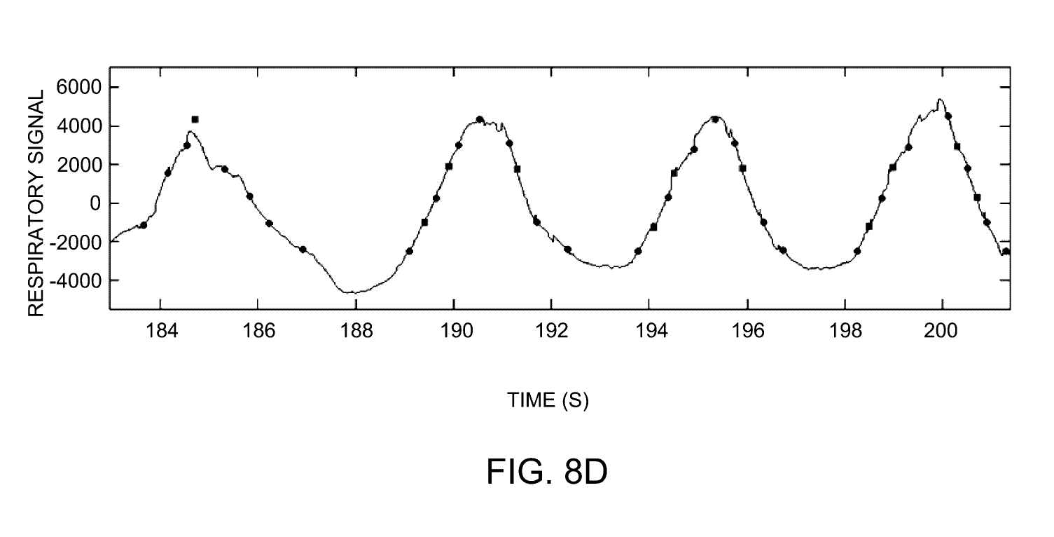

FIGS. 8A-8D show the acquisition simulation results of the greedy acquisition method. FIGS. 8A-8C show the triggering process in each of the three healthy volunteers. Each dot represents an image acquisition trigger position and the waveform is the volunteer's respiratory signal. As with the order prefixed acquisition methods in FIGS. 6A-6D and 7A-7D, the end of inhalation and the end of exhalation state amplitudes were calculated using 20 seconds preparation stage signals. FIG. 8D shows a subset of FIGS. 8A-8C to further illustrate missed triggers within the greedy acquisition method. Each circle is a successful trigger. Each square represents a missed trigger. The total scan time of the three volunteers was 642 s, 651 s, and 492 s respectively for the greedy acquisition method (excluding the scanner adjustment and respiration preparation stages).

By using the greedy acquisition method, the scan halts caused by trigger missing were removed in the example experiment and the scan time was reduced. The total scan time for T2 weighted 4DMRI was limited to a reasonable range for clinical use.

The greedy acquisition method does not follow a fixed slice and state acquisition order during the scan. The slice and state acquisition order depend on the breathing pattern. Although each missed trigger may not cause any scan halt, the missed acquisition may need to be triggered later. Most of the missed triggers occur in the middle of the breathing, the states triggered at the end of an acquisition mainly concentrated at states 40% ↑, 60% ↑, 80% ↑, 80% ↓, 60% ↓, and 40% ↓, as can be seen in FIGS. 8A-8C.

Because the shot time for each slice acquisition was fixed, the minimum number of the missed triggers within each respiratory cycle may be determined. Accordingly, some missed triggers may be unavoidable. For example, if acquiring the 40% ↑ state results in the missing of the following 60% ↑ state trigger in one respiratory cycle, one of these two triggers may be given up in that cycle no matter how the acquisitions are arranged. The greedy algorithm facilitates reducing the triggers missed in one respiratory cycle, even if the 4DMRI system cannot select which trigger to give up. The greedy algorithm automatically acquires the current available trigger. In the above example, the greedy algorithm always chooses 40% ↑ state trigger since it is the first to reach. The 60% ↑ state trigger is left for future acquisition. The choice of missed triggers may affect the total scan time.

The greedy acquisition method may also increase the trigger efficiency at the end of exhalation state. Because of the relatively slow breathing at the end of exhalation state, more than one slice at that state may be triggered during each respiratory cycle. FIGS. 8A-8C shows that the acquisitions of slices at the end of exhalation state were finished much earlier than the acquisitions of other states. After all the slices had been acquired at the end of exhalation state, there was no image acquisition triggered even when the respiratory signal reached the end of exhalation state trigger. Since the end of exhalation state generally lasts longer than other respiratory states, the end of exhalation state is used as a gating state in treatment machine gating radiotherapy. In some embodiments, the greedy acquisition method may acquire further sets of images at the end of exhalation state during a 4DMRI scan to improve the signal to noise ratio (SNR) of the end of exhalation state without using extra scan time.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A magnetic resonance imaging (MRI) system comprising:
a sensor configured to detect a characteristic amplitude of a subject, the characteristic amplitude associated with a patient characteristic of the subject;
a receiver configured to receive magnetic resonance data;
a memory device; and
a processor programmed to:
determine when the characteristic amplitude of the subject reaches a first characteristic amplitude of a plurality of sequential characteristic amplitudes defined for a cycle of the patient characteristic of the subject;
determine if a predetermined number of MRI images have been acquired for the first characteristic amplitude;
capture magnetic resonance data with the receiver if the number of MRI images have not been acquired for the first characteristic amplitude and without regard for a location of the first characteristic amplitude in the sequence of the plurality of sequential characteristic amplitudes; and
process the magnetic resonance data as an MRI image associated with the first characteristic amplitude.

2. The MRI system of claim 1, wherein the patient characteristic is the respiration of the subject and the cycle is a respiratory cycle of the subject, the characteristic amplitude of the subject is a respiratory amplitude, the plurality of sequential characteristic amplitudes is a plurality of sequential respiratory amplitudes, and the first characteristic amplitude is a first respiratory amplitude.

3. The MRI system of claim 2, wherein the MRI image is a T2 weighted MRI image.

4. The MRI system of claim 2 further comprising a respiratory belt including the sensor, the respiratory belt configured to engage the body of the subject, wherein the sensor is a pressure sensor configured to detect the respiratory amplitude of the subject based on a pressure associated with the respiratory belt.

5. The MRI system of claim 2, wherein the processor is further programmed to:
determine that the predetermined number of MRI images have been acquired for the first respiratory amplitude; and
in response to the determination, prevent additional magnetic resonance data associated with the first respiratory amplitude from being captured, wherein magnetic resonance data is captured for remaining respiratory amplitudes of the plurality of sequential respiratory amplitudes until the predetermined number of MRI images have been acquired for each of the remaining respiratory amplitudes.

6. The MRI system of claim 2, wherein the plurality of sequential respiratory amplitudes comprises predetermined sequential respiratory states of the respiratory cycle of the subject, wherein the processor is further programmed to detect the respiratory amplitude of the subject in order of the predetermined sequential respiratory states.

7. The MRI system of claim 6, wherein the predetermined sequential respiratory states include an end of inhalation state, an end of exhalation state, and at least one respiratory state between the end of inhalation state and the end of exhalation state.

8. A method for acquiring a predetermined number of magnetic resonance imaging (MRI) images at each of a plurality of sequential characteristic amplitudes of a subject for four dimensional (4D) MRI, where each characteristic amplitude is an amplitude within a cycle of a patient characteristic of a subject, the method comprising:
monitoring, by a processor, the characteristic amplitude of the subject;
detecting when the monitored characteristic amplitude reaches a first characteristic amplitude of the plurality of sequential characteristic amplitudes;
capturing magnetic resonance data for the first characteristic amplitude if the predetermined number of MRI images have not been acquired for the first characteristic amplitude and without regard for a location of the first characteristic amplitude in the sequence of the plurality of sequential characteristic amplitudes; and
processing, by the processor, the magnetic resonance data as an MRI image associated with the first characteristic amplitude.

9. The method of claim 8, wherein the patient characteristic is the respiration of the subject and the cycle is a respiratory cycle of the subject, the characteristic amplitude of the subject is a respiratory amplitude, the plurality of sequential characteristic amplitudes is a plurality of sequential respiratory amplitudes, and the first characteristic amplitude is a first respiratory amplitude.

10. The method of claim 9, wherein the MRI image is a T2 weighted MRI image.

11. The method of claim 9, wherein monitoring the characteristic amplitude of the subject further comprises:
coupling a respiratory belt to the body of the subject, the respiratory belt including a pressure sensor; and
monitoring a pressure associated with the respiratory belt to detect the respiratory amplitude of the subject.

12. The method of claim 9 further comprising:
determining, by the processor, that the predetermined number of MRI images have been acquired for the first respiratory amplitude; and
in response to the determination, preventing additional magnetic resonance data associated with the first respiratory amplitude from being captured, wherein magnetic resonance data is captured for remaining respiratory amplitudes of the plurality of sequential respiratory amplitudes until the predetermined number of MRI images have been acquired for each of the remaining respiratory amplitudes.

13. The method of claim 9, wherein the plurality of sequential respiratory amplitudes comprises predetermined sequential respiratory states of the respiratory cycle of the subject, wherein monitoring the characteristic amplitude of the subject further comprises detecting the respiratory amplitude of the subject in order of the predetermined sequential respiratory states.

14. The method of claim 13, wherein the predetermined sequential respiratory states include an end of inhalation state, an end of exhalation state, and at least one respiratory state between the end of inhalation state and the end of exhalation state.

15. At least one non-transitory computer-readable storage media having computer-executable instructions embodied thereon, wherein when executed by at least one processor, the computer-executable instructions cause the processor to:
determine when a characteristic amplitude of a subject reaches a first characteristic amplitude of a plurality of sequential characteristic amplitudes defined for a cycle of a patient characteristic of the subject;
determine if a predetermined number of MRI images have been acquired for the first characteristic amplitude;
capture magnetic resonance data if the predetermined number of MRI images have not been acquired for the first characteristic amplitude and without regard for a location of the first characteristic amplitude in the sequence of the plurality of sequential characteristic amplitudes; and
process the magnetic resonance data as an MRI image associated with the first characteristic amplitude.

16. The computer-readable storage media of claim 15, wherein the patient characteristic is the respiration of the subject and the cycle is a respiratory cycle of the subject, the characteristic amplitude of the subject is a respiratory amplitude, the plurality of sequential characteristic amplitudes is a plurality of sequential respiratory amplitudes, and the first characteristic amplitude is a first respiratory amplitude.

17. The computer-readable storage media of claim 16, wherein the MRI image is a T2 weighted MRI image.

18. The computer-readable storage media of claim 16, wherein the computer-executable instructions further cause the processor to monitor the respiratory amplitude of the subject using a pressure sensor of a respiratory belt, the respiratory belt engaging the body of the subject, wherein the pressure sensor is configured to detect the respiratory amplitude of the subject based on a pressure associated with the respiratory belt.

19. The computer-readable storage media of claim 16, wherein the computer-executable instructions further cause the processor to:
determine that the predetermined number of MRI images have been acquired for the first respiratory amplitude; and
in response to the determination, prevent additional magnetic resonance data associated with the first respiratory amplitude from being captured, wherein magnetic resonance data is captured for remaining respiratory amplitudes of the plurality of sequential respiratory amplitudes until the predetermined number of MRI images have been acquired for each of the remaining respiratory amplitudes.

20. The computer-readable storage media of claim 16, wherein the plurality of sequential respiratory amplitudes comprises predetermined sequential respiratory states of the respiratory cycle of the subject, wherein the processor is further programmed to detect the respiratory amplitude of the subject in order of the predetermined sequential respiratory states.
